(19) United States
(12) Patent Application Publication
(10) Pub. No.: US 2016/0082040 A1
(43) Pub. Date: Mar. 24, 2016

(54) NITRIC OXIDE TREATMENT OF MAMMARY TISSUE

(71) Applicant: Bovicor Pharmatech Inc., Vancouver (CA)

(72) Inventors: Christopher C. Miller, North Vancouver (CA); Gilly Regev-Shoshani, Vancouver (CA)

(73) Assignee: Bovicor Pharmatech Inc., Vancouver (CA)

(21) Appl. No.: 14/829,435

(22) Filed: Aug. 18, 2015

Related U.S. Application Data
(60) Provisional application No. 62/038,619, filed on Aug. 18, 2014.

(51) Int. Cl. A61K 33/00; A61M 5/31; A61M 13/00; A61K 9/00

(52) U.S. Cl. CPC A61K 33/00; A61K 9/0014; A61M 5/3129; A61M 13/00; A61M 2202/0275; A61M 2250/00

(57) ABSTRACT

Systems, compositions, devices, and methods for treating a mammary condition in a mammal using gNO delivered from a nitric oxide releasing solution (NORS) are disclosed and described. In one embodiment, the mammary condition may be mastitis.

### PRIORITY DATA

This application claims the benefit of U.S. Provisional Patent Application Ser. No. 62/038,619 filed on Aug. 18, 2014, which is incorporated herein by reference.

### FIELD OF TECHNOLOGY

The present technology relates to compositions, dosage forms and associated methods for treating a mammary condition in a mammal. Accordingly, invention embodiments involve the fields of chemistry, pharmaceutical sciences, veterinary sciences, medicine and other health sciences.

### BACKGROUND

Mastitis is a mammary condition typically characterized by inflammation of the mammary gland. Mastitis is often caused by bacterial infection. In mild cases, mastitis can cause localized pain, swelling and redness in the mammary gland and surrounding tissue. Further, if left untreated, mastitis can quickly progress into a more severe condition with systemic effects, including fever, mild-depression, and other flu-like symptoms.

Mastitis can be especially problematic in the dairy industry. Once present in a cow, mastitis can quickly spread to other cows through routine milking activities and can present great cost to dairy farmers in reduced salable milk production and treatment cost. As a result, dairy farmers employ various means to treat and prevent mastitis, including both topical and internal measures. Teat dips are commonly used pre- and post-milking in an attempt to disinfect the skin surrounding the teat sphincter. However, once the teat cistern becomes infected with bacteria, teat dips become essentially ineffective at treating the condition. To treat such an infection, dairy farmers often use a syringe to directly inject an antibiotic into the teat cistern. After direct injection of antibiotics, milk must be withheld for several milking periods and the animal cannot be used for meat production for at least several days and possibly up to a month following administration, thus presenting a greatly negative economic impact.

### SUMMARY OF INVENTION EMBODIMENTS

Invention embodiments include methods of treating a mammary condition in a subject. In one embodiment such a method can include administering a therapeutically effective amount of gaseous nitric oxide (gNO) to the subject's mammary tissue. In one aspect, such administration can be achieved by administering gNO from a nitric oxide releasing solution or substance (NORS) to an external mammary tissue or an internal mammary tissue, or both.

Additionally, certain invention embodiments encompass a system for administering a therapeutically effective amount of gNO to a subject's mammary tissue. Such a system can in some embodiments, include a first component configured to release gNO upon acidification and a second component configured to acidify the first component. Such a system can include a device for holding and administered the first and second components of the system.

Further invention embodiments provide methods for minimizing an interruption in usable milk production by a cow in order to treat the cow for a mammary condition. In one embodiment, such a method may include administering to the cow's mammary tissue, a therapeutically effective amount of gNO from a NORS solution.

There has thus been outlined, rather broadly, exemplary invention features so that the detailed description thereof that follows may be better understood, and so that the present contribution to the art may be better appreciated. Other invention features will become clearer from the following detailed description taken with the accompanying drawings and claims.

### BRIEF DESCRIPTION OF THE DRAWINGS

Invention features and advantages will be apparent from the detailed description which follows, and are further enhanced in conjunction with the accompanying drawings, which together illustrate, by way of example, various invention embodiments; and, wherein:

FIG. 1 is a schematic view of a bovine mammary gland.

FIG. 2 is a schematic view of an embodiment of a device for intramammary infusion (i.e. administration) of a NORS solution.

FIG. 3 shows the amount of gNO detected at 3, 8, and 15 min as well as 3, 4 (2A) and 24 (2B) hours. The X scale is TIME (minutes) from start on measuring point (showing pre-measuring amount as 0-0.1ppm) and Y scale showing amount of gNO (measured in ppb).

FIG. 4 is a graph representing the amount of milk production (L) from all 3 treated animals before, during, and one day post study.

FIG. 5 is a chart representing the amount of nitrites found in serum after 5, 30 and 480 min post treatment. Treatment was 100, 200 and 400 mM of nitrites at pH 3.9. Animals' numbers are 1111, 1108 and 1064. * means significantly higher than baseline.

FIG. 6 is a chart representing the amount of nitrites found in milk at 8 and 24 hours post treatment. Treatment was 100, 200 and 400mM of nitrites at pH 3.9. Animals' numbers are 1111, 1108 and 1064.

A—FR/FL/BR/BL are the 4 quarters, where the quarter that was treated is circled in red.

B—large milk batch from machine milking, 8 hours post Tx.

C—stripped milk after machine milking was completed, from the treated quarter.

24 hours—sample taken from treated quarter, 24 hours post Tx.

FIG. 7 is a chart representing the MetHg(%) measured in blood samples taken 5, 30 and 480 min post treatment (400 mM).

FIG. 8 is a chart showing E. coli counts in 400 mM samples at ratios of 1:2, 1:1, and 2:1 milk to nitrite solution.

These drawings are provided to illustrate various aspects certain invention embodiments and are not intended to be limiting in scope in terms of dimensions, materials, configurations, arrangements or proportions unless otherwise limited by the claims.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this specification and the appended claims, the singular forms “a,” “an” and “the” include plural referents unless the context clearly dictates otherwise. Thus, for example, reference to “a teat” includes a plurality of teats.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

Occurrences of the phrase “in one embodiment,” or “in one aspect,” herein do not necessarily all refer to the same embodiment or aspect.

As used herein, “subject” refers to a mammal that may benefit from the administration of NORS. In one aspect the mammal may be a bovine. In another aspect, the bovine may be a cow.

As used herein, the terms “treat,” “treatment,” or “treating” when used in conjunction with the administration of NORS, including compositions and dosage forms thereof, refers to administration to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Compositions can take nearly any physical state, including solid, liquid (i.e. solution), or gas. Furthermore, the term “dosage form” can include one or more formulation(s) or composition(s) provided in a format for administration to a subject. In one example, a composition can be a solution that releases nitric oxide.

As used herein “NORS” refers to a nitric oxide (NO) releasing solution, composition or substance. In one aspect, NO released from NORS may be a gas.

As used herein a “therapeutic agent” refers to an agent that can have a beneficial or positive effect on a subject when administered to the subject in an appropriate or effective amount. In one aspect, NO can be a therapeutic agent.

As used herein, an “effective amount” of an agent is an amount sufficient to accomplish a specified task or function desired of the agent. A “therapeutically effective amount” of a composition, drug, or agent refers to a non-toxic, but sufficient amount of the composition, drug, or agent, to achieve therapeutic results in treating or preventing a condition for which the composition, drug, or agent is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician, veterinarian, or other qualified medical personnel using evaluations known in the art, it is recognized that individual variation and response to treatments may make the achievement of therapeutic effects a somewhat subjective decision. The determination of an effective amount or therapeutically effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986).

As used herein, a “dosing regimen” or “regimen” such as “treatment dosing regimen,” or a “prophylactic dosing regimen” refers to how, when, how much, and for how long a dose of a composition can or should be administered to a subject in order to achieve an intended treatment or effect.

As used herein, the terms “release” and “release rate” are used interchangeably to refer to the discharge or liberation, or rate thereof, of a substance, including without limitation a therapeutic agent, such as NO, from the dosage form or composition containing the substance. In one example, a therapeutic agent may be released in vitro. In another aspect, a therapeutic agent may be released in vivo.

As used herein, “immediate release” or “instant release” can be used interchangeably and refer to immediate or near immediate (i.e. uninhibited or unrestricted) release of an agent or substance, including a therapeutic agent, such as NO, from a composition or formulation.

As used herein, the term “controlled release” refers to non-immediate release of an agent or substance, including a therapeutic agent, such as NO, from a composition or formulation. Examples of specific types of controlled release include without limitation, extended or sustained release and delayed release. Any number of control mechanisms or components can be used to create a controlled release effect, including formulation ingredients or constituents, formulation properties or states, such as pH, an environment in which the formulation is placed, or a combination of formulation ingredients and an environment in which the formulation is placed. In one example, extended release can include release of a therapeutic agent at a level that is sufficient to provide a therapeutic effect or treatment for a non-immediate specified or intended duration of time.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 ml to about 80 ml” should also be understood to provide support for the range of “50 ml to 80 ml.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually, and further including decimal or fraction values such as 1.8, 2.3, 3.7, and 4.2.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Reference in this specification may be made to devices, structures, systems, or methods that provide “improved” performance. It is to be understood that unless otherwise stated, such “improvement” is a measure of a benefit obtained based on a comparison to devices, structures, systems or methods in the prior art. Furthermore, it is to be understood that the degree of improved performance may vary between disclosed embodiments and that no equality or consistency in the amount, degree, or realization of improved performance is to be assumed as universally applicable.

### Example Embodiments

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Varieties of mammary conditions affect mammals and reduce their ability to produce milk for their offspring or for commercial use. One such condition is mastitis, which is characterized as an inflammation of mammary tissue. Mastitis can cause mild to severe discomfort in a subject and significantly reduce milk production for commercial and non-commercial uses. Some examples of infecting organisms include S. aureus, S. epidermis, streptococci and many other microorganisms. In addition to mastitis, there are many other mammary conditions that affect mammary tissue, such as mammary herpes, mammary tuberculosis, mammary syphilis, mammary actinomycosis, poxvirus infection, dermatitis, warts, duct ectasia, and various mammary abscesses caused by the above listed and other conditions. Furthermore, treatments of these conditions can require that the milk be discarded from commercial and non-commercial use for a period of one or more days. In the case of commercial subjects, treatments can also prevent use of the animal for meat production for a significant time period.

Invention embodiments relate to formulations, systems and methods for treating a mammary condition in a subject. One embodiment includes a method of administering a therapeutically effective amount of gaseous nitric oxide (gNO) to the mammary tissue of a subject. gNO can be administered to an internal tissue, an external tissue or both. Some examples of internal tissues include mammary cisterns, such as the papilla or teat cistern and gland cistern, the milk ducts, the lobules and alveoli. External tissues can include the mammary papilla and adjacent skin surrounding the mammary papilla. gNO can be administered utilizing a variety of formulations. In one embodiment gNO is administered as part of, or from, a nitric oxide releasing solution or substance (NORS). A NORS is a vehicle for releasing gNO, including therapeutically effective amounts thereof, to the site of administration and/or to a targeted treatment site that is distal to the administration site. Administration of the NORS provides for the quick delivery of the NORS to the targeted treatment site, followed by an extended and prolonged release of gNO at the treatment site, or a location distal thereto. It is noted that gNO can be present as a dissolved gas and/or it can be free from solution.

In one embodiment, the NORS can include the use of water or a saline-based solution or substance and at least one NO releasing compound, such as nitrite or a salt thereof. In one embodiment, the NORS is a saline-based solution or substance. In one embodiment, the NO releasing compound is a nitrite, a salt thereof, or any combinations thereof Non-limiting examples of nitrites include nitrite salts such as sodium nitrite, potassium nitrite, barium nitrite, and calcium nitrite, mixed salts of nitrite such as nitrite orotate, and nitrite esters such as amyl nitrite. In one embodiment, the NO releasing compound is selected from the group consisting of sodium nitrite and potassium nitrite, or any combinations thereof. In another embodiment, the NO releasing compound is sodium nitrite. In one embodiment, the NORS can comprise a sodium nitrite in a saline solution. In another embodiment, the solution can comprise a potassium nitrite in a saline solution. In one embodiment, the concentration of NO releasing compound, for example, nitrite (i.e. NO₂), in the NORS can be from 0.07% w/v to about 2.0 w/v. In another embodiment the concentration of nitrites in the solution can be from 0.07% w/v to about 1.0% w/v. In another embodiment, the concentration of nitrites in the solution can be from about 1.0% w/v to about 2.0% w/v. In another embodiment, the concentration of nitrites in the solution can be from about 1.0% w/v to about 1.5% w/v. In another embodiment, the concentration of nitrites in the solution can be from about 1.5% w/v to about 2.0% w/v. In one embodiment, the concentration of nitrites in the solution is no greater than about 0.5% w/v. In another embodiment, the concentration of nitrites in the solution is about 0.1% w/v. In a further embodiment, the concentration of nitrites in the solution is about 0.2% w/v. In an additional embodiment, the nitrite concentration is about 0.3% w/v. In another embodiment, the nitrite concentration is about 0.4% w/v. In yet another embodiment, the concentration of nitrite in the solution is about 0.28% w/v. In an additional embodiment, the nitrite concentration in the solution is about 0.32% w/v. In an additional embodiment, the nitrite concentration in the solution is about 0.38% w/v. In another embodiment, the nitrite concentration in the solution is about 0.41% w/v. In a further embodiment, the nitrite concentration in the solution is about 0.46% w/v. In a further embodiment, the nitrite concentration in the solution is about 0.92% w/v. In a further embodiment, the nitrite concentration in the solution is about 1.84% w/v. In a further embodiment, the nitrite concentration in the solution is about 2.30% w/v. In a further embodiment, the nitrite concentration in the solution is about 2.76% w/v. In a further embodiment, the nitrite concentration in the solution is about 3.22% w/v. In another embodiment, the nitrite concentration in the solution is from about 0.07% w/v to about 0.5% w/v. In a further embodiment, the nitrite concentration in the solution can be from about 0.05% w/v to about 10% w/v. As used herein, the term “w/v” refers to the (weight of solute/volume of solution)×100%. In one embodiment, when sodium nitrite is used in the solution, the concentration of sodium nitrite can be from about 0.41% w/v to about 0.69% w/v. In another embodiment, when sodium nitrite is used in the solution, the concentration of sodium nitrite can be from about 0.3% w/v to about 3.0% w/v. In another embodiment, when sodium nitrite is used in the solution, the concentration of sodium nitrite can be from about 0.3% w/v to about 1.0% w/v. In another embodiment, when sodium nitrite is used in the solution, the concentration of sodium nitrite can be from about 1.0% w/v to about 1.5% w/v. In another embodiment, when sodium nitrite is used in the solution, the concentration of sodium nitrite can be from about 1.5% w/v to about 2.0% w/v. In another embodiment, when sodium nitrite is used in the solution, the concentration of sodium nitrite can be from about 2.0% w/v to about 2.5% w/v. In another embodiment, when sodium nitrite is used in the solution, the concentration of sodium nitrite can be from about 2.5% w/v to about 3.0% w/v.

In an additional embodiment, the amount of NO releasing agent, for example nitrite (i.e. NO₂), can be a concentration of from about 1 mM to about 1 M. In another embodiment, the nitrite concentration can be from about 10 mM to about 500 mM. In another embodiment, the nitrite concentration can be from about 50 mM to about 400 mM. In another embodiment, the nitrite concentration can be from about 100 mM to about 400 mM. In yet a further embodiment, the nitrite concentration in the solution can be from about 100 mM to about 200 mM. In another embodiment, the nitrite concentration in the solution can be from about 200 mM to about 300 mM. In an additional embodiment, the nitrite concentration in the solution can be from about 300 mM to about 400 mM. In another embodiment, the nitrite concentration in the solution can be from about 400 mM to about 500 mM. In an additional embodiment, the nitrite concentration in the solution can be from about 40 mM to about 180 mM. In a further embodiment, the nitrite concentration in solution can be about 160 mM. In an additional embodiment, the nitrite concentration in solution can be from about 40 mM to about 120 mM. In another embodiment, the nitrite concentration can be about 60 mM. In yet another embodiment, the nitrite concentration can be about 100 mM. In an additional embodiment, the nitrite concentration can be about 200 mM. In an additional embodiment, the nitrite concentration can be about 300 mM. In an additional embodiment, the nitrite concentration can be about 400 mM. In an additional embodiment the concentration of nitrite in the solution can be about 109 mM or less. In a further embodiment, when sodium nitrite is used in the solution, the concentration of sodium nitrite can be about 72 mM.

In one embodiment, the NORS can also contain at least one acidifying agent. As described elsewhere herein, the addition of at least one acidifying agent to the NORS solution contributes toward increased production (ie. attenuates production) of NO from the NORS solution or substance. Any acidifying agent which contributes to NO production is contemplated by the present technology. In one embodiment, the acidifying agent can be an acid. In one aspect, the acid can be an organic acid. In another aspect, the acid can be an inorganic acid. Non-limiting examples of acids include ascorbic acid, salicylic acid, malic acid, lactic acid, citric acid, formic acid, benzoic acid, tartaric acid, carbonic acid, hydrochloric acid, sulfuric acid, nitric acid, nitrous acid and phosphoric acid. In one embodiment, the acid is selected from the group consisting of ascorbic acid, citric acid, malic acid, hydrochloric acid, and sulfuric acid, or any combinations thereof. In another embodiment, the acid is citric acid. Alternatively, the acidifying agent can include an acidifying gas such as NO, N₂O, NO₂, CO₂, and other acidifying gases. In one aspect, the acidifying gas may be NO. In another aspect, the acidifying agent can be an acidifying solid, such as alginic acid, an acidified gelatin, polyacrylic acid, and other acidifying solids. In addition, acidifying agents may include compounds or molecules that produce or release an acid, including any of the aforementioned acids, upon addition to the NORS solution.

As described above, the amount of acidifying agent present in the solution can affect the rate of the reaction to produce NO. In one embodiment, the amount of acidifying agent is no greater than about 5.0% w/v of the solution. In another embodiment, the amount of acidifying agent is about 0.5% w/v. In another embodiment, the amount of acidifying agent is about 0.2% w/v. In a further embodiment, the amount of acidifying agent is about 0.07% w/v. In another embodiment, the amount of acidifying agent is between about 0.07-5.0% w/v. In another embodiment, the amount of acidifying agent is between about 0.07-10.0% w/v. In another embodiment, the amount of acidifying agent is between about 5.0-10.0% w/v.

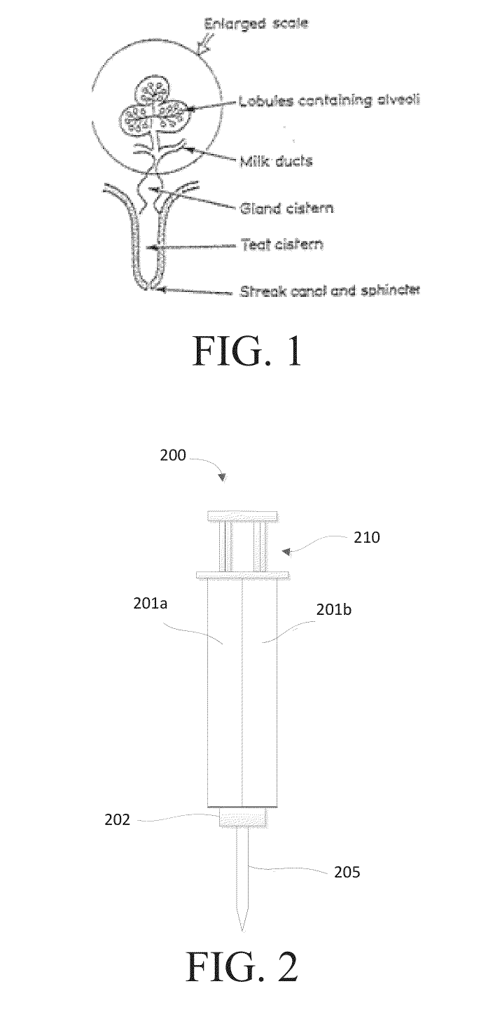

In one embodiment, the solution may be administered to the subject via intramammary injection or can be applied topically, or both. Referring to FIG. 1 is shown a schematic representation of a bovine mammary structure. In an embodiment, where the NORS is administered via intramammary delivery, the NORS solution will enter the teat through the teat orifice or sphincter and proceed into the teat cistern. The NORS solution may travel to other parts of the mammary tissue, such as the gland cistern, milk ducts, and lobules, depending on the volume administered. In some embodiments, the NORS solution may enter the teat through the orifice or sphincter and the gNO released from the solution will permeate to the other parts of the mammary tissue.

Nearly any device capable of administering NORS into the teat can be used. In many cases, a device, such as a syringe with a long extended portion that can penetrate through the teat orifice or sphincter to some degree is helpful in administering the NORS into the teat cistern. One exemplary device such as a double barrel syringe is shown in FIG. 2. The double barrel syringe is labeled generally as 200. In use, one barrel 201a of the syringe housing can hold the portion of the NORS containing the nitric oxide producing components and the other barrel 201b of the housing can hold the acidifying agent. Upon depressing the plunger 210, the nitric oxide producing components can be compelled toward a mixing portion 202 of the syringe and the mixed components can be eluted via the syringe tip 205. Accordingly, upon injecting the syringe contents into the teat orifice or sphincter, the components from each barrel mix and the NORS solution is acidified. In other aspects, separate administration devices can be used to administer the NORS and the acidifier separately, where upon the NORS becomes acidified in-vivo. In yet other embodiments, the NORS solution can be acidified at or prior to administration and the acidified solution administered to the subject from a single barrel syringe or other suitable or single device. Again, nearly any mechanism or device capable of delivering the NORS in a manner that allows release of NO in-vivo can be employed.

As previously mentioned, in some embodiments, the formulation may be an immediate release formulation. In other embodiments, the formulation can be a controlled release formulation which releases g NO for an extended period of time. In some embodiments, the carrier of the solution may be water, or a saline solution. In other embodiments, other carriers can be used.

In use, the NORS can release a therapeutically effective concentration of gNO. In one embodiment, the therapeutically effective concentration of gNO is between about 4 ppm and about 10,000 ppm. In one embodiment, the therapeutically effective concentration of gNO is between about 4 ppm and about 5000 ppm. In one embodiment, the therapeutically effective concentration of gNO is between about 4 ppm and about 1000 ppm. In one embodiment, the therapeutically effective concentration of gNO is between about 1000 ppm and about 10,000 ppm. In one embodiment, the therapeutically effective concentration of gNO is between about 1000 ppm and about 5,000 ppm. In one embodiment, the therapeutically effective concentration of gNO is between about 5000 ppm and about 10,000 ppm. In one embodiment, the therapeutically effective concentration of gNO is between about 500 ppm and about 1000 ppm. In one embodiment, the therapeutically effective concentration of gNO is between about 100 ppm and about 500 ppm. In another embodiment, the therapeutically effective concentration of gNO is between about 40 ppm and about 400 ppm. In another embodiment, the therapeutically effective concentration is between about 50 and 200 ppm. In another embodiment, the therapeutically effective concentration is about 160 ppm.

A NORS can be prepared as a single phase or multi-phase formulation. In one embodiment the NORS can be formulated as a two-phase or two-part composition that includes at least one nitrite or salt thereof in a first part of the two-part composition, and an acidifying agent in the second part of the two-part composition. The acidifying agent can be a liquid, gas, or solid as previously mentioned.

When administering a NORS and/or a gNO to an internal tissue, in some embodiments, the NORS can be prepared a liquid solution that can be delivered as a solution or as an aerosol. In some aspects, a liquid solution can minimize the stress caused to the subject upon administration of the NORS and throughout the treatment period. If administering the NORS to a skin surface, other carriers may be used.

In one embodiment, the NORS can be characterized as having a multiple states of activity, such as a dormant state and an active state. Furthermore, the active state may have one or more sub-states where release rate or activity of the NORS varies. As contemplated herein, the dormant state of the NORS is one in which the pH of the solution or substance is above 5.0 and exhibits a minimal or undetectable production level of nitric oxide gas. In one embodiment, the pH of the dormant state of the NORS is between a pH of about 5.0 and a pH of about 7.0. The active state of the NORS is one in which the pH of the solution is below about 5.0 and exhibits an increased or enhanced production level of nitric oxide gas, including production at a therapeutically effective level, and in some embodiments, over an extended period of time. In one embodiment, the pH of the active state of the NORS is between a pH of about 1.0 and a pH of about 5.0 and may include a number of sub-states characterized by amount or rate of gNO release. In another embodiment, the pH of the active state of the NORS is between a pH of about 3.0 and a pH of about 5.0. In one embodiment, the pH is about 3.2. In another embodiment the pH is about 4.0. In another embodiment, the pH is about 3.9. In another embodiment, the pH is about 3.8 In another embodiment, the pH is about 3.7. In another embodiment, the pH is about 3.6. In yet another embodiment, the pH is about 3.5. In yet a further embodiment, the pH is about 3.4. In an additional embodiment, the pH is about 3.3. In yet another embodiment, the pH can be from about 3.0 to about 3.5. Because the NORS of the present invention can have multiple states of activity or inactivity, the solution or substance can be pre-made, transported and set up for administration while in its dormant state (pH greater than 4.0), without losing any appreciable amount of gNO or without losing its ability to produce a therapeutically effective amount of gNO. Once a user is ready to deliver or administer the solution or substance to a subject, the solution or substance can be activated prior to administration to the subjection, for example, immediately prior (pH driven below 4.0), thereby maximizing the amount of gNO produced by the administered dosage of NORS. In an alternative embodiment, the NORS can be acidified beforehand and administered at a later time. In some embodiments a mechanism or device for storing or otherwise formulating an acidified NORS can be used which preserves its potency and therapeutic effect when administered.

For example, by introducing sodium nitrite (or other salts of nitrites) to a saline solution it will very slowly produce nitric oxide gas, but in an undetectable amount (as measured by chemiluminescence analysis methodology (ppb sensitivity)). The rate of NO produced from the solution increases as pH decreases. The rate of gNO production increases significantly once the solution's pH is below 4.5. In one embodiment, where an acidifying gas is used to lower the NORS pH, such as an acidifying NO gas, an unexpected result is that the amount of NO gas evolving from (i.e. coming out of) the NORS is more than the amount of NO gas added. Generally, NO is produced based on the following equilibrium equations:

NO₂⁻+H⁺→HNO₂ 1.

2HNO₂→N₂O₃+H₂O→H₂O+NO+NO₂ 2a.

3HNO₂⇄2NO+NO₃⁻+H₂O+H⁺ 2b.

The acid (e.g. from the acidifying agent) donates the H⁺ to the nitrite (NO₂⁻). The amount of H⁺ present, affects the rate at which the reaction moves towards HNO₂. Additionally, NO production is facilitated by HNO₂ concentration. As can be seen from these equations, increasing the concentration of nitrites (ie. NO releasing compound or agent) present in the NORS (for example 60 mM versus 20 mM), requires more acid to achieve the same pH. In other words, the more HNO₂ produced, the lower the pH will be. NO production rate and/or amount can be increased by either increasing the H+ or the (NO2-) concentration. Interestingly, either NO₂ or pH alone, even at optimal levels, is insufficient to have an antimicrobial, or therapeutic effect on a subject.

In one example embodiment, citric acid may be used as an acidifying agent. In such embodiment, a pH of from about 3.45 to about 3.65 can be achieved in saline solution including about 20 mM nitrites (about 0.14% w/v) by adding a sufficient amount of citric acid to achieve a concentration of about 7.3 mM (about 0.14% w/v). At a higher concentration of nitrites, such as 60 mM nitrites (about 0.4% w/v), a sufficient amount of citric acid can be added to the saline solution to achieve a concentration of 15.6 mM (about 0.3% w/v) in order to reduce the pH to between about 3.45 and 3.65. At a concentration of 100 mM nitrites (about 0.7% w/v), a sufficient amount of citric acid can be added to the saline solution to achieve a concentration of 36 mM (about 0.7% w/v) in order to reduce the pH to between about 3.45 and 3.65. The pH range of 3.45 to 3.65 is merely one example of a useful pH range in the current technology. As will be apparent to those skilled in the art, pH ranges higher than 3.45 to 3.65 can be achieved by decreasing the amount acidifying agent in the composition. Alternatively, pH ranges lower than 3.45 to 3.65 can be achieved by increasing the amount of acidifying agent in the composition. For example, in a volume of 40 mL a pH of about 3.9 can be achieved at a concentration of 50 mM nitrites by adding approximately 45 mg citric acid to achieve a concentration of acidifying agent of about 5.75 mM. In another example, in a volume of 40 mL a pH of about 3.9 can be achieved at a concentration of 100 mM nitrites by adding approximately 79 mg citric acid to achieve a concentration of acidifying agent of about 10.25 mM. In another example, in a volume of 40 mL a pH of about 3.9 can be achieved at a concentration of 200 mM nitrites by adding approximately 144 mg citric acid to achieve a concentration of acidifying agent of about 18.75 mM. In another example, in a volume of 40 mL a pH of about 3.9 can be achieved at a concentration of 400 mM nitrites by adding approximately 273 mg citric acid to achieve a concentration of acidifying agent of about 36.25 mM.

In yet another example embodiment, ascorbic acid can be used as an acidifying agent. Specifically, a sufficient amount of ascorbic acid can be added to a saline solution including 20 mM nitrites (about 0.14% w/v) to achieve a concentration of 127 mM (about 2.25% w/v) ascorbic acid in order to reduce the pH to between about 3.45 and 3.65. At a higher concentration of nitrites, such as 60 mM nitrites (about 0.4% w/v), a sufficient amount of ascorbic acid can be added to the saline solution to achieve a concentration of 352 mM (about 6.2% w/v) in order to reduce the pH to between about 3.45 and 3.65. At a concentration of 100 mM nitrites (about 0.7% w/v), a sufficient amount of ascorbic acid can be added to the saline solution to achieve a concentration of 545 mM (about 9.6% w/v) in order to reduce the pH to between about 3.45 and 3.65. The pH range of 3.45 to 3.65 is merely one example of a useful pH range in the current technology. As will be apparent to those skilled in the art, pH ranges higher than 3.45 to 3.65 can be achieved by decreasing the amount acidifying agent in the composition. Alternatively, pH ranges lower than 3.45 to 3.65 can be achieved by increasing the amount of acidifying agent in the composition.

Accordingly, an effective amount of acidifying agent can be from about 5 mM to about 40 mM. In another aspect, the effective amount of acidifying agent can be from about 1 mM to about 1000 mM. In another aspect, the effective amount of acidifying agent can be from about 1 mM to about 100 mM. In another aspect, the effective amount of acidifying agent can be from about 1 mM to about 15 mM. In another aspect, the effective amount of acidifying agent can be from about 5 mM to about 20 mM. In another aspect, the effective amount of acidifying agent can be from about 15 mM to about 30 mM. In another aspect, the effective amount of acidifying agent can be from about 30 mM to about 50 mM. In another aspect, the effective amount of acidifying agent can be from about 50 mM to about 200 mM. In another aspect, the effective amount of acidifying agent can be from about 150 mM to about 300 mM. In another aspect, the effective amount of acidifying agent can be from about 250 mM to about 500 mM. In another aspect, the effective amount of acidifying agent can be from about 400 mM to about 1000 mM.

In some embodiments, the NORS can be configured or prepared to provide an initial burst or dose of gNO, followed by an extended release thereof at a therapeutically effective amount. In some embodiments, the gNO release can be according to a desired or pre-designed profile. In one embodiment, the profile can specify the release of gNO in an amount that is the equal to the amount released initially over the course of the period. In another aspect the profile can specify release of gNO in an amount that decreases as compared to the amount released initially over the course of the period. In some embodiments, the decrease may be from 10% to 90% over the specified period. In other embodiments, the decrease may be from about 30% to about 80% over the specified period. In a further aspect, the decrease may be from about 40% to about 70% over the specified period. In an additional aspect, the decrease may be about 50% over the specified period. In some embodiments, the period can be from about 5 minutes to about 1 week. In another embodiment, the specified period can be from about 1 day to about 1 week. In a further embodiment the specified period can be from about 5 minutes to about 24 hours. In yet an additional embodiment, the specified period can be from about 5 minutes to about 12 hours. In another embodiment, the specified period can be from about 5 minutes to about 1 hour. In another embodiment, the specified period can be from about 5 minutes to about 30 minutes.

The NORS may be administered to the subject as a controlled or extended release formulation of gNO, and optionally with a carrier formulation. In one aspect, the extended release may release an effective amount of gNO from the formulation at a controlled rate such that therapeutically effective levels (but below toxic levels) of the component are maintained over an extended period of time. In one aspect, the period of time may range from about 1 minute to about 24 hours. In another aspect, the period of time can range from about 30 to about 60 minutes. In another aspect, the time period may be several hours. In another embodiment, the period of time can be from about 10 to about 45 minutes. In yet a further embodiment, the time period may be at least 15 minutes. In one embodiment, the time period can be at least 30 minutes. In one embodiment, the time period can be at least 8 hours. In another embodiment, the time period can be at least 12 hours. In an additional embodiment the time period can correspond to an interval between milk expression events by the subject, such as milking or feeding of offspring. In one aspect, such interval or period may be about or at least 4 hours, about 8 hours, about 12 hours, from about 2 hours to about 12 hours, or other suitable time period. Thus, the administered NORS provides for continuous or otherwise extended delivery of NO to the treatment site of the subject, or a location distal therefrom. Further, the amount of administered NORS may be varied in order to optimize the duration of NO production and delivery.

Some invention embodiments provide a method of treating a subject by delivering a NORS to a treatment site of the subject and allowing gNO to be produced by the NORS. In one aspect, the amount of gNO released is a therapeutically effective amount. In some aspects, the amount or NORS delivered may be selected in order to allow a specific amount of gNO to be released. Any disease, disorder, or condition where NO delivery is beneficial or provides a therapeutic effect can be treated. Exemplary mammary conditions include but are not limited to, mastitis, mammary herpes, mammary tuberculosis, mammary syphilis, mammary actiomycosis, poxvirus infection, dermatitis, warts, duct ectasia, and various mammary abscesses. In certain embodiments, the NORS can be prepared just prior to administration (e.g. within 5 or 10 minutes) to the subject by activating a dormant NORS with an acidifying agent. For example, as described elsewhere herein, an organic acid may be added to the dormant NORS, such as citric acid. In other embodiments, an acidifying gas, such as NO or NO₂ containing gas can be used. Once the acidifier is added to the dormant NORS the NORS is activated and can be administered to the subject. As previously mentioned, the activated NORS can provide for extended production of NO. Further, in some embodiments, the acidifying agent can be added from about 30 seconds to about 2 hours prior to administration of the NORS to a subject. In another aspect, the acidifying agent can be added from about 1 minute to about 1 hour prior to administration of the NORS to a subject. In an additional aspect, the acidifying agent can be added from about 4 to about 48 hours prior to administration. In a further aspect, the acidifying agent may be added at administration of the NORS to a subject. In a further embodiment, the acidifying agent may be added following administration of the NORS, or of a gNO releasing compound or agent to a subject. In this embodiment, the NORS solution is formed in-vivo and releases gNO thereafter.

In some embodiments, the NORS may be reapplied (ie administered) one or more times, as necessary to effectively treat the subject. Administration to mammary tissue can occur as a single dose or as multiple doses at one or more specified internal locations as part of a treatment regimen. In one aspect the interval can be about once every day. In another aspect, the interval can be about once every 7 days. In another aspect, the interval can be about once every 14 days. In a further aspect, the interval can be about once every 28 days. In another aspect, the interval can be from about every hour to every 28 days. Other suitable intervals can also be used, such as every 1-12 months. Nearly any interval identified as particularly effective in treating a subject can be used.

In one embodiment, the NORS can be administered post-milking or nursing so as to administer the NORS in an environment substantially free of milk and other bodily fluids. In one aspect, the administration can be within 1 hour following milking. In another embodiment, the administration can be within 30 minutes following milking. In a further aspect, the administration can be within 15 minutes following milking. In yet another aspect, the administration can be immediately following milking. In some embodiments, the milking process can include stripping of the teat. In some embodiments, the NORS can be configured to attain an in-vivo pH which allows the NORs to be active and provide gNO release at a specifically desired rate or amount over time as the production of milk in the mammary tissue continues and the pH of the in-vivo environment changes.

Alternatively, the NORS can be administered in the presence of milk or other bodily fluids in a manner that compensates for the presence of those fluids and still maintains a pH sufficient to keep the solution active and provide therapeutically effective gNO production. In another embodiment, a NORS can be administered to an external mammary tissue as part of a treatment regimen either by itself or in combination with intramammary administered NO. In one aspect, such topical administration can occur in connection with a milking event. NORS can be applied pre-milking or nursing, post-milking or nursing, or both. Regimens for application of NORS to internal and external mammary tissues can be performed together or separately. The amount of NORS administered can depend on administration to an internal or external surface. Generally, an amount of NORS administered to an internal mammary tissue will be the same or smaller than an amount administered to an external surface. However, in some circumstances the amount of NORS delivered to an internal mammary tissue can be greater than the amount delivered to an external mammary tissue. The amount administered will also depend on species, physical development of the subject, severity of the condition, and other factors. In one embodiment, the amount of NORS administered to an internal mammary tissue is between about 0.25 ml and about 50 ml per mammary papilla. In one embodiment, the amount of NORS administered to an internal mammary tissue is between about lml and about 45 ml per mammary papilla. In another embodiment, the NORS administered to an internal mammary tissue is between about 5 ml and about 50 ml per mammary papilla. In a further embodiment, the amount is about 10 ml per mammary papilla. In another embodiment, the amount is about 20 ml per mammary papilla. In another embodiment, the amount is about 30 ml per mammary papilla. In another embodiment, the amount is about 40 ml per mammary papilla. In one embodiment, the amount of NORS administered to an external mammary tissue is between about 0.5 and about 100 ml per mammary papilla. In one embodiment, the amount of NORS administered to an external mammary tissue is between about 1 and about 40 ml.

NORS can be administered in any suitable frequency. In one aspect, NORS can be administered as a single dose treatment. In one aspect, NORS can be administered in multiple doses. As previously discussed, administration of NORS can be made in connection with a milking event. Treatment can occur at a single milking event. However, treatment can be made in 2 or more milking events. These treatments can be made in connection with successive milking events, such as 2-3 or more successive milking events. The treatments can also be spread out over a period of days or weeks, such as once per day for 2-3 or more successive days, once every other day for 2-3 or more successive intervals, every tree days for 2-3 or more successive intervals, once per week for 2-3 successive weeks, once every two weeks for 2-3 successive intervals, or any other suitable treatment regimen. Additionally, the treatments need not be made in connection with a milking event. For example, treatments can be made in connection with, or prior to, drying off the subject or applying teat sealant. Additionally, treatments can be made while the subject is dry. Treatments can be made to address an immediate condition or they can be made prophylactically.

The present technology allows for delivery of NO to an ambulatory subject. For example, the extended production and delivery of NO to the treatment site by way of the administered NORS allows for the treated subject to remain ambulatory during treatment. Thus, the subject is not constrained to a nitric oxide delivery device during the entire duration of NO delivery. The present invention provides methods of treatment in any suitable subject, including primates, cattle, horses, dogs, cats, pigs, sheep, goats, camels, buffalos, and other mammals.

In one embodiment, the method includes treatment of a mammary condition in a subject. Exemplary mammary conditions treated by way of the present methods include, but are not limited to mastitis, mammary herpes, mammary tuberculosis, mammary syphilis, mammary actiomycosis, poxvirus infection, dermatitis, warts, duct ectasia, various mammary abscesses and the like. In one embodiment, a method includes treatment of a mammary condition in a subject, including infections caused by a virus, a fungus, a protozoan, a parasite, an arthropod or a bacterium, including a bacterium that has developed resistance to one or more antibiotics. In certain embodiments, the method can include the treatment of a mammary condition or disorder caused by a bacterial or viral invention. In one embodiment, the NORS can be administered when a disease state is present. In one embodiment, the NORS can be administered prophylactically in order to prevent onset of a disease state.

As mentioned, treatment of a mammary condition by way of the present invention can include the delivery of a NORS to an internal or external mammary tissue of the subject to be treated. For example, in certain embodiments, the NORS can be injected, sprayed, instilled or otherwise applied to an internal mammary tissue of the subject. In certain embodiments the NORS can be sprayed, wiped, rubbed, dipped or otherwise topically applied to an external mammary tissue of the subject. The NORS may be administered to an internal mammary tissue by intramammary delivery, such as by inserting a device for delivering the NORS a sufficient distance into the orifice or sphincter of the mammary papilla and injecting a sufficient volume of NORS within the mammary canal or cistern. In one embodiment, the NORS is sprayed into the mammary canal or cistern of the subject using a spray device. In one embodiment, the NORS is injected within the mammary canal or cistern using a syringe or other similar device.

Whenever a therapeutic agent is delivered to an internal mammary tissue of an animal, there is concern about potential systemic effects and associated health risks. Delivery of a NORS to an internal mammary tissue of a subject can lead to systemic increase in blood nitrite levels. However, an increase in MetHg(%) in the blood of less than 10% is within safety limits. Introduction of a NORS to an internal mammary tissue, according to the current technology, does not cause an increase in MetHg(%) in the blood of the subject of 10% or more. In one embodiment, introduction of a NORS to an internal mammary tissue of a subject can induce an increase in MetHg(%) of less than 8%. In one embodiment, introduction of a NORS to an internal mammary tissue of a subject can induce an increase in MetHg(%) of less than 7%. In one embodiment, introduction of a NORS to an internal mammary tissue of a subject can induce an increase in MetHg(%) of less than 6%. In one embodiment, introduction of a NORS to an internal mammary tissue of a subject can induce an increase in MetHg(%) of less than 5%. In another embodiment, blood MetHg(%) levels induced by administration of NORS can return to baseline within a period of up to 480 minutes post administration. In another embodiment, blood MetHg(%) levels induced by administration of NORS can return to baseline within a period of up to 360 minutes post administration. In another embodiment, blood MetHg(%) levels induced by administration of NORS can return to baseline within a period of up to 240 minutes post administration. In another embodiment, blood MetHg(%) levels induced by administration of NORS can return to baseline within a period of up to 120 minutes post administration.

Additionally, delivery of a NORS to an internal mammary tissue of a subject can lead to an increase in serum nitrite levels. In one embodiment, serum nitrite levels induced by administration of the NORS return to baseline within a period of up to 480 minutes post administration. In one embodiment, serum nitrite levels induced by administration of the NORS return to baseline within a period of up to 360 minutes post administration. In one embodiment, serum nitrite levels induced by administration of the NORS return to baseline within a period of up to 240 minutes post administration. In one embodiment, serum nitrite levels induced by administration of the NORS return to baseline within a period of up to 120 minutes post administration.

One embodiment of the current technology includes a method of treating a mammary condition in a bovine subject. The method involves administering a NORS to the subject's mammary tissue that provides an amount of gNO that is therapeutically effective in treating the mammary condition, but where serum nitrate levels are maintained at a reasonably low level. In one aspect of this method, serum nitrite levels remain below 7500 nM. In another aspect, serum nitrite levels remain below 6500 nM. In another aspect, serum nitrite levels remain below 5000 nM. In another aspect, serum nitrite levels remain below 4000 nM. In another aspect, serum nitrite levels remain below 3000 nM. In another aspect, serum nitrite levels remain below 2000 nM.

Another embodiment of the current technology describes a method of treating a mammary condition in a bovine subject. The method involves administering a NORS to the subject's mammary tissue that provides an amount of gNO that is therapeutically effective in treating the mammary condition, but where blood MetHg(%) levels are maintained at a reasonably low level. In one aspect, the blood MetHg(%) of the subject remains below 10%. In another aspect, the blood MetHg(%) of the subject remains below 8%. In another aspect, the blood MetHg(%) of the subject remains below 5%. Introducing a therapeutic agent to an internal mammary tissue can raise concerns about the viability of the milk product obtained from the subject, whether for use with the subject's offspring or for commercial use. In some cases, residual therapeutic agent can be found in the milk for several days or weeks post-treatment, rendering the milk unusable or unsalable. However, NO has many qualities, including a short half-life in the body, that give it the potential to be an effective therapeutic for treating a mammary condition without long-lasting adverse consequences. Accordingly, the current technology also includes a method for minimizing an interruption in usable or salable milk production by a subject due to treatment of the subject for a mammary condition. The method can include administering a therapeutically effective amount of gNO from a NORS solution to the subject's mammary tissue. gNO can increase nitrite levels in the milk product, but it is generally only of very short duration and generally only interrupts usable or salable milk from the treated mammary papilla. In one embodiment, the interruption occurs for less than 6 milking events. In one embodiment, the interruption occurs for less than 4 milking events. In one embodiment, the interruption occurs for 1 milking event. In one embodiment, the interruption is for a period of up to 48 hours. In one embodiment, the interruption is for a period of up to 24 hours. In one embodiment, the interruption is caused by residual nitrites, nitrates, or both in the milk product. In one embodiment, residual nitrites in the milk return to baseline levels within a period of up to 48 hours. In one embodiment, residual nitrites in the milk return to baseline levels within a period of up to 24 hours. In one embodiment, residual nitrates in the milk return to baseline levels within a period of up to 48 hours. In one embodiment, residual nitrates in the milk return to baseline levels within a period of up to 24 hours. In one embodiment, both residual nitrites and residual nitrates in the milk return to baseline levels within a period of up to 48 hours. In one embodiment, both residual nitrites and residual nitrates in the milk return to baseline levels within a period of up to 24 hours. In one embodiment, the interruption occurs only in the treated mammary papilla. In one embodiment, the subject is a cow. In one embodiment, the subject is a bovine cow. In one embodiment, the mammary condition is mastitis.

Additionally, infusing a therapeutic agent into a teat or delivering a therapeutic agent to an internal mammary tissue can raise concerns about curdling of the milk product. However, NORS can be administered to an internal mammary tissue of a subject without curdling the milk product of the subject and still maintain a therapeutic effect to the subject. More specifically, a NORS with 200 mM nitrites at pH 3.8 can cause a 70% reduction in bacterial counts in milk without curdling the milk product at a ratio of 1:1 NORS to milk product. In another aspect, a NORS with 400 mM nitrites at pH 4 can cause a complete eradication of bacterial counts in milk without curdling the milk product at a ratio of either 1:1 or 1:2 NORS to milk product.

Another embodiment of the present technology can include a method of treating a mammary condition in a bovine subject. This method includes administering a NORS to the subject's mammary tissue that provides an amount of gNO that is therapeutically effect in threating the mammary condition, but where milk nitrite levels return to near-baseline levels within a 24-hour period. In one aspect, milk nitrite levels return to less than 200% of baseline within a 24-hour period. In one aspect, milk nitrite levels return to less than 175% of baseline within a 24-hour period. In one aspect, milk nitrite levels return to less than 150% of baseline within a 24-hour period. In one aspect, milk nitrite levels return to less than 125% of baseline within a 24-hour period. Baseline levels can be determined at a time prior to administering the NORS to the subject's mammary tissue. For example, baseline levels can be determined at a regular milking event just prior to administration of the NORS. In one aspect, baseline levels can be determined at any time within 12 hours prior to administration (i.e. 12 hours, 10 hours, 8 hours, 4 hours, 2 hours, 1 hour, 30 minutes, 10 minutes, or 5 minutes before administration of the NORS). In general, the baseline is intended to represent the normal value of nitrites in the subject's milk prior to treatment with the NORS.

Another embodiment of the present technology includes a method of treating a mammary condition in a bovine subject. The method can include administering a NORS to the subject's mammary tissue that is compositionally configured to provide an amount of gNO that is therapeutically effective in treating the mammary condition when the NORS is administered into the mammary tissue.

Another embodiment of the present technology includes a method of treating a mammary condition in a bovine subject. The method includes removing substantially all of the milk out of the mammary quarter of the bovine subject and then administering a NORS to the subject's mammary quarter that provides an amount of gNO that is therapeutically effective in treating the mammary condition. Removing substantially all of the milk out of the mammary quarter of the bovine can be done by any suitable method. In one aspect, removing all of the milk includes manually stripping the milk out of the quarter after routine hand or machine milking. In one aspect, removing all of the milk can include administering a facilitating agent to help remove substantially all of the milk from the mammary quarter. In one aspect, the facilitating agent can be any suitable galactagogue. The galactagogue can be natural, synthetic, and/or herbal. Galactagogues can include oxytocin, and other hormones. Galactagogues can include goat's rue, milk thistle, fennel, fenugreek, shatavari, and other herbal galactagogues. Galactagogues can include risperidone, chlorpromazine, and other synthetic compounds. In one particular aspect, the facilitating agent can be oxytocin.

Another embodiment of the present technology can include a method of treating a mammary condition in a bovine subject. The method can include pre-conditioning a mammary quarter of the bovine subject and administering a NORS to the subject's mammary quarter that provides an amount of gNO that is therapeutically effective in treating the mammary condition. Pre-conditioning can include administering a sterile solution to either an internal and/or an external mammary tissue of the mammary quarter. In one aspect, the sterile solution can be a sterile saline solution. In one aspect, the sterile solution can be an acidified saline solution. In one aspect, the sterile solution can be an acidifying agent.

Another embodiment of the present technology includes a system for treating a mammary condition in a bovine subject. The system can include a pre-condition solution and a NORS. The NORS can provide an amount of gNO that is therapeutically effective in treating the mammary condition. In one aspect, the pre-conditioning solution can be a sterile solution. In one aspect, the pre-conditioning solution can be a saline solution. In one aspect, the pre-conditioning solution can be an acidified saline solution. In one aspect, the pre-conditioning solution can be an acidifying solution.

Another embodiment of the present technology can include a system for storing, transporting, handling, and/or administering a NORS solution and/or for performing the administration of gNO, as described above. In one embodiment, the system can include a first component configured to release gNO upon acidification thereof and a second component configured to acidify the first component. The first component can include at least one nitrite or salt thereof. The second component can include any acidifying agent, whether gaseous, liquid, or solid. Such acidifying agents can include, nitric oxide gas, carbon dioxide gas, nitrous acid, carbonic acid, phosphoric acid, alginic acid, gelatin, polyacrylic acid, and any other suitable acidifying agent.

The first and second components can be combined to form an activated formulation. In one embodiment, the pH of the activated formulation is below about 5.0. In one embodiment, the pH of the activated formulation is between about 3.0 and about 5.0. In one embodiment, the pH of the activated formulation is between about 3.2 and about 3.9. In another embodiment, the pH of activated formulation is between about 3.2 and about 4.2. In one embodiment, the pH of the activated formulation is about 3.6. In one embodiment, the pH of activated formulation is about 3.7. In one embodiment, the pH of activation is about 3.8. In one embodiment, the pH of activation is about 3.9. In one embodiment the pH of activation is about 4.0. In one embodiment, the pH of the activated formulation is lower that the target pH in order to compensate for the presence of milk or other fluids. In these formulations, the pH can be from about 1.0 to about 3.0 or from about 1.5 to about 2.5. Once the formulation is activated upon combining the first and second components, the activated formulation can release gNO in an amount and/or at a rate that achieves a concentration of from about 40 to about 10,000 ppm gNO at the treatment site, or at a site distal therefrom. In one embodiment, the activated formulation can release an amount that provides a gNO concentration of from about 60 to about 200 ppm. In an additional embodiment, the activated formulation can release gNO for a period of about or at least 0.25, 0.5, 1, 2, 4, 6, 8, and 12 hours.

The system can also include a device for combining and/or administering the first and second components. In one embodiment, the device is configured to administer the first and second components to an internal mammary tissue by delivering the components within the mammary cistern or canal. Such a device can include a syringe, multi-channel syringe, a spray device, a multi-channel spray device, or other suitable delivery device. As previously mentioned, one exemplary device is shown generally in FIG. 2. In one aspect, the first and second components can be mixed as they are dispensed from a multi-channel syringe or spray device. In one aspect, the first and second components can be added to a single-channel syringe, single-channel spray device, or other suitable device in which the components can be mixed in the same chamber and subsequently administered. In another aspect, the first and second components can be administered to the subject separately at the same or similar situs and become mixed in situ (e.g. in vivo) by any suitable means. In another embodiment, the device is configured to administer the first and second components to an external mammary tissue via topical administration. This can be accomplished by mixing the first and second components in a dipping basin for submersion of the mammary papilla and surround skin in the basin subsequent to mixing the components. In another aspect, the second component can be a thickening or gelling agent that is capable of acidifying the first component, such as alginic acid, which is mixed with the first component to form a gel, ointment, or cream that can be wiped, rubbed or otherwise applied to the external mammary tissue. In another aspect, the first component can be applied to the external mammary surface and the second component can be subsequently sprayed or otherwise applied to the external mammary surface for in situ mixing. There are many other ways of mixing and applying the first and second components of the system which will be apparent to those skilled in the art, and such methods of application are contemplated as part of the present technology. In one embodiment, the systems and methods contemplated herein may include both intramammary and topical administration of gNO to a subject, particularly from a NORS.

Another embodiment of the current technology can include a dose of a NORS that is effective for treating a mastitis condition in a bovine when administered thereto. The dose can include an effective amount of a gNO releasing compound or solution. Additionally, the dose can include an effective amount of an acidifying agent or solution. In one aspect, the gNO releasing compound or solution and the acidifying agent or solution can be mixed to produce a NORS at a pH of between 3.0 and 5.0. In one aspect, the pH is between 3.2 and 4.5. In one aspect, the pH is between 3.4 and 4.2. In one aspect. The pH is between 3.4 and 3.6. In one aspect, pH is between 3.6 and 4.1. In one aspect, the pH is between 3.7 and 4.0. In one aspect, the pH is about 3.4. In one aspect, the pH is about 3.5. In one aspect, the pH is about 3.6. In one aspect, the pH is about 3.7. In one aspect, the pH is about 3.8. In one aspect, the pH is about 3.9. In one aspect, the pH is about 4.0. In one aspect, the pH is about 4.1. In one aspect, the dose of NORS is pre-mixed. In one aspect, the dose of NORS is not pre-mixed prior to administration.

In one aspect, the volume of the dose is less than 50 mL. In one aspect, the volume of the dose is between 30 mL and 50 mL. In one aspect, the volume of the dose is between 35 mL and 45 mL. In one aspect, the dose is between 10 mL and 20 mL. In one aspect, the dose is between 20 mL and 30 mL. In one aspect, the dose is between 30 mL and 40 mL. In one aspect, the dose is between 40 mL and 50 mL.

In one aspect, the dose or dosage of NORS can provide a therapeutically effective concentration of gNO to the subject. In one embodiment, the concentration of gNO provided or released by the dose or dosage is between about 4 ppm and about 10,000 ppm. In one embodiment, the concentration of gNO is between about 4 ppm and about 5000 ppm. In one embodiment, the concentration of gNO is between about 4 ppm and about 1000 ppm. In one embodiment, the concentration of gNO is between about 1000 ppm and about 10,000 ppm. In one embodiment, the concentration of gNO is between about 1000 ppm and about 5,000 ppm. In one embodiment, the concentration of gNO is between about 5000 ppm and about 10,000 ppm. In one embodiment, the concentration of gNO is between about 500 ppm and about 1000 ppm. In one embodiment, the concentration of gNO is between about 100 ppm and about 500 ppm. In another embodiment, the concentration of gNO is between about 40 ppm and about 400 ppm. In another embodiment, the concentration is between about 50 and 200 ppm. In another embodiment, the concentration is about 160 ppm.

In one aspect, the dose can include an effective amount of a nitric oxide releasing solution. The effective dose can include from about 50 mM to about 400 mM nitrites. In another aspect, effective dose can include from about 1 mM to about 1 M nitrites. In another embodiment, the effective dose can include from about 10 mM to about 500 mM nitrites. In another embodiment, the effective dose can include from about 100 mM to about 400 mM nitrites. In yet a further embodiment, the effective dose can include from about 100 mM to about 200 mM nitrites. In another embodiment, the effective dose can include from about 200 mM to about 300 mM nitrites. In an additional embodiment, the effective dose can include from about 300 mM to about 400 mM nitrites. In another embodiment, the effective dose can include from about 400 mM to about 500 mM nitrites. In an additional embodiment, the effective dose can include from about 40 mM to about 180 mM nitrites. In a further embodiment, the effective dose can include about 160 mM nitrites.

In an additional embodiment, the effective dose can include from about 40 mM to about 120 mM nitrites. In another embodiment, the effective dose can include about 60 mM nitrites. In yet another embodiment, the effective dose can include about 100 mM nitrites. In an additional embodiment, the effective dose can include about 200 mM nitrites. In an additional embodiment, the effective dose can include about 300 mM nitrites. In an additional embodiment, the effective dose can include about 400 mM nitrites. In an additional embodiment the effective dose can include about 109 mM or less of nitrites. In a further embodiment, when sodium nitrite is used in the solution, the effective dose can include about 72 mM.

In one aspect the dose can include an effective amount of acidifying agent to produce a concentration in the dose of from about 5 mM to about 40 mM. In another aspect, the effective amount of acidifying agent can be from about 1 mM to about 1000 mM. In another aspect, the effective amount of acidifying agent can be from about 1 mM to about 100 mM. In another aspect, the effective amount of acidifying agent can be from about 1 mM to about 15 mM. In another aspect, the effective amount of acidifying agent can be from about 5 mM to about 20 mM. In another aspect, the effective amount of acidifying agent can be from about 15 mM to about 30 mM. In another aspect, the effective amount of acidifying agent can be from about 30 mM to about 50 mM. In another aspect, the effective amount of acidifying agent can be from about 50 mM to about 200 mM. In another aspect, the effective amount of acidifying agent can be from about 150 mM to about 300 mM. In another aspect, the effective amount of acidifying agent can be from about 250 mM to about 500 mM. In another aspect, the effective amount of acidifying agent can be from about 400 mM to about 1000 mM.

The current technology can be illustrated by a number of non-exclusive example embodiments as follows:

In one example, a method of treating a mammary condition in a subject is described, which comprises administering a therapeutically effective amount of gaseous nitric oxide (gNO) to the subject's mammary tissue from a nitric oxide releasing solution (NORS).

In one example, the mammary tissue is an internal tissue, including mammary cisterns, milk ducts, lobules and alveoli.

In one example, the mammary tissue is an external tissue, including mammary papilla and adjacent skin.

In one example, the NORS is prepared from a two-part composition, a first part of the two-part composition including at least one nitrite or salt thereof and a second part of the two-part composition including an acidifying agent.

In one example, NORS is a liquid solution.

In one example, the NORS includes at least one nitric oxide releasing compound in an amount of less than about 1.0% w/v.

In one example, the NORS is administered in an amount from about 0.25 mL to about 50 mL.

In one example, the NORS is administered in a regimen of about once every 28 days.

In one example, the NORS is administered in a regimen of about once every 14 days.

In one example, the NORS is administered in a regimen of about once every 7 days.

In one example, the NORS is prepared within about 10 minutes of administration to the mammal.

In one example, the NORS is prepared within about 5 minutes of administration to the mammal.

In one example, the NORS is administered post-milking or post-nursing.

In one example, mammary condition is at least one of mastitis, mammary herpes, mammary tuberculosis, mammary abscess, mammary syphilis, poxvirus infection, dermatitis, warts, mammary actinomycosis, and duct ectasia.

In one example, the mammary condition is mastitis.

In one example, administration occurs when disease state is present.

In one example, administration is prophylactic.

In one example, administration is intramammary delivery.

In one example, delivery is performed using a device selected from the group consisting of a spray device and a syringe.

In one example, intramammary delivery is performed using a syringe.

In one example, the syringe is a double-barreled syringe.

In one example, administration is topical.

In one example, the therapeutically effective amount of gNO is a concentration of from about 50 ppm to about 400 ppm.

In one example, the therapeutically effective amount of gNO is a concentration of about 160 ppm.

In one example, the duration of administration is from about 1 hour to about 28 days.

In one example, serum nitrite levels induced by administration of the NORS return to baseline within a period of up to 480 minutes post administration.

In one example, blood MetHg(%) levels induced by administration of NORS return to baseline within a period of up to 480 minutes post administration.

In one example, a system for treating a mammary condition in a subject, as recited in any of the previous examples, is described, which comprises a first component configured to release gNO upon acidification thereof; a second component configured to acidify the first component; and a device for administering the first and second component such that when combined, the components form a composition that releases a therapeutically effective amount of gNO to the subject.

In one example, the system further comprises a device for combining the first and second components either prior to, during, or following administration to the subject.

In one example, the device is configured to administer the first and second component within a mammary cistern.

In one example, the device is configured to administer the first and second component topically.

In one example, the first and second components are combined prior to administration.

In one example, the first and second components are combined upon administration.

In one example, the first and second components are combined following administration.

In one example, the first component includes at least one nitrite or salt thereof and the second component includes an acidifying agent.

In one example, the first and second component are combined to form an activated composition.

In one example, the volume of activated composition is from about 10 to about 50 ml.

In one example, the pH of activation is lower than about 5.0.

In one example, the pH of activation is from about 3.2 to about 4.2.

In one example, the pH of activation is about 3.9.

In one example, the pH of activation is about 3.7.

In one example, the pH of activation is lower than a target pH to compensate for milk or other fluids present.

In one example, the activated formulation releases gNO at a concentration of from about 50 to about 10,000 ppm.

In one example, the activated formulation releases gNO at a concentration of from about 60 to about 200 ppm.

In one example, the activated formulation releases gNO for a period of about 1 hour to about 12 hours.

In one example, the activated formulation releases gNO for a period of about 8 hours.

In one example, the activated formulation releases gNO for a period from about 0.25 to about 4 hours.

In one example, a method for minimizing an interruption in usable milk production by a cow due to treatment of the cow for a mammary condition is described, which comprises administering to the cow's mammary tissue, a therapeutically effective amount of gNO from a NORS solution.

In one example, the interruption occurs for less than 6 milking events.

In one example, the interruption occurs for less than 4 milking events.

In one example, the interruption occurs for 1 milking event.

In one example, the mammary condition is mastitis.

In one example, the interruption is for a period of up to 48 hours.

In one example, the interruption is for a period of up to 24 hours.

In one example, a method of treating a mammary condition in a bovine subject is described, which comprises administering a NORS to the subject's mammary tissue that provides an amount of gNO that is therapeutically effective in treating the mammary condition, wherein serum nitrite levels remain below 7500 nM.

In one example, serum nitrite levels remain below 6500 nM.

In one example, serum nitrite levels remain below 5000 nM.

In one example, serum nitrite levels remain below 3000 nM.

In one example, serum nitrite levels remain below 2000 nM.

In one example, a method of treating a mammary condition in a bovine subject is described, which comprises administering a NORS to the subject's mammary tissue that provides an amount of gNO that is therapeutically effective in treating the mammary condition, wherein blood MetHg(%) remains below 10%.

In one example, blood MetHg(%) remains below 8%.

In one example, blood MetHg(%) remains below 5%.

In one example, a method of treating a mammary condition in a bovine subject is described, which comprises administering a NORS to the subject's mammary tissue that provides an amount of gNO that is therapeutically effective in treating the mammary condition, wherein milk nitrite levels return to less than 200% of a baseline value within a 24 hour period.

In one example, the milk nitrite levels return to less than 175% of a baseline value within a 24 hour period.

In one example, the milk nitrite levels return to less than 150% of a baseline value within a 24 hour period.

In one example, the milk nitrite levels return to less than 125% of a baseline value within a 24 hour period.

In one example, a method of treating a mammary condition in a bovine subject is described, which comprises removing substantially all of the milk out of a mammary quarter of the bovine subject; and administering a NORS to the subject's mammary quarter that provides an amount of gNO that is therapeutically effective in treating the mammary condition.

In one example, removing substantially all of the milk out of a mammary quarter includes administering an effective amount of a facilitating agent to the subject.

In one example, the facilitating agent is oxytocin.

In one example, a method of treating a mammary condition in a bovine subject is described, which comprises pre-conditioning a mammary quarter of the bovine subject; and administering a NORS to the subject's mammary quarter that provides an amount of gNO that is therapeutically effective in treating the mammary condition.

In one example, pre-conditioning includes administering a volume of a saline solution to the mammary quarter of the bovine subject.

In one example, pre-conditioning includes administering a volume of an acidifying solution to the mammary quarter of the bovine subject.

In one example, a system for treating a mammary condition in a bovine subject is described, which comprises a pre-conditioning solution; and a NORS that provides an amount of gNO that is therapeutically effective in treating the mammary condition.

In one example, the pre-conditioning solution is a saline solution.

In one example, the pre-conditioning solution is an acidifying solution.

In one example, a method treating a mammary condition in a bovine subject is described, which comprises administering a NORS to the subject's mammary tissue which is compositionally configured to provide an amount of NO that is therapeutically effective in treating the mammary condition when the NORS is administered into the mammary tissue.

In one example, a dose of a NORS that is effective for treating a mastitis condition in a bovine when administered thereto is described, which comprises an effective amount of a gNO releasing compound; and an effective amount of an acidifying agent, wherein the gNO releasing compound and the acidifying agent are mixed to produce the NORS at a pH of between 3.2 and 4.5.

In one example, the volume of the dose is less than 50mL.

In one example, the volume of the does is about 40mL.

In one example, the NORS is pre-mixed.

In one example, the NORS is not pre-mixed.
In one example, the effective amount of gNO releasing solution contains from about 50 mM to about 400 mM nitrates.
In one example, the effective amount of acidifying agent is from about 5 mM to about 50 mM.
In one example, the pH is from about 3.7 to about 4.0.
In one example, the pH is 3.9.

### EXAMPLES

Certain invention embodiments are further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Extended Release of NO from NORS

An experiment was performed to evaluate gNO production from the above-recited NORS solution. A NORS solution was prepared with a saline base and a nitrite concentration of 0.3% w/v and pH 3.7. Once ready, a 3×3 in gauze was dipped into the solution, lightly squeezed to discard excess liquid and placed in a “Hath Bath” device. At different time points, the NO that was being released was measured with a chemiluminescence analyzer (NOA 280i, General Electric, CO).

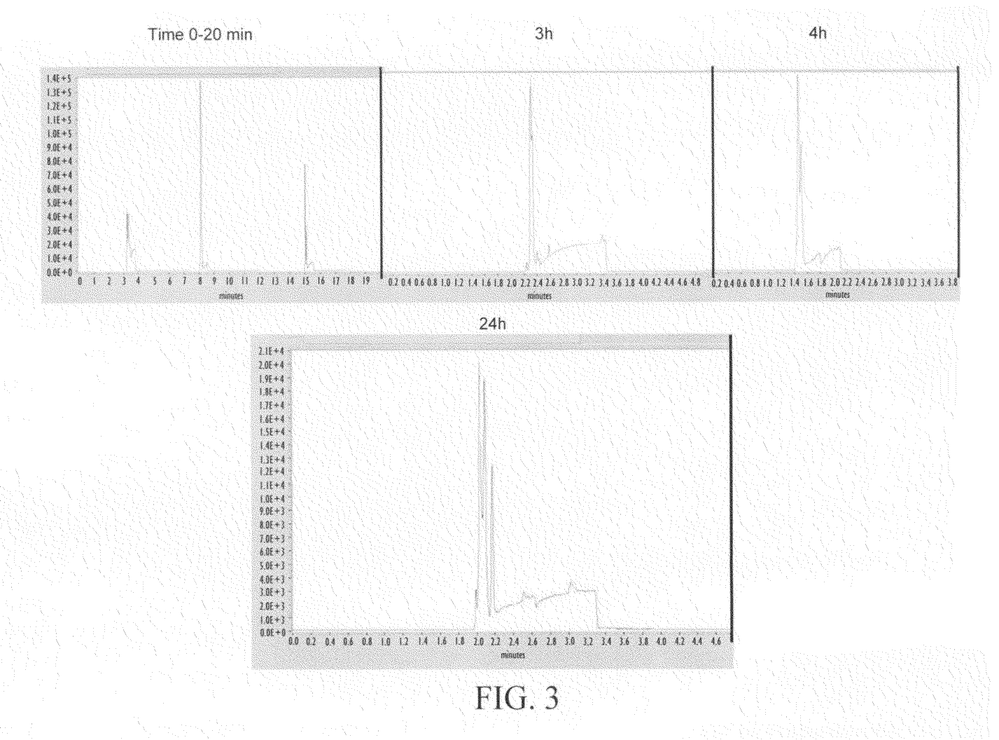

FIG. 3 shows the amount of NO detected at 3, 8, 15 min as well as 3, 4 (2A) and 24 (2B) hours. The X scale is TIME (minutes) from start on measuring point (showing pre-measuring amount as 0-0.1ppm) and Y scale showing amount of NO (measured in ppb).

The chemiluminescence analyzer has a sample draws rate of 200 cc per min and thus, there is an initial peak and reduction in NO concentration following that. The “Hathback” may not be completely sealed and thus some NO may “escape”. However, release of NO was still detected 24 hours after gauze was saturated with the NORS solution.

### Example 2

### In-vitro Antibacterial Effects of NORS

The effect of a NORS on various bacterial strains is evaluated. A mono-microbial infection tissue model developed by Barry University (Miami Shores, FL) that allows evaluation of the effects of NORS in reducing bacterial load using full thickness skin is employed. Skin is supplied by MatTek Corporation with Staphyloccous aureus, Escheria coli, Methicillin-resistant Staphyloccous aureus (MRSA), Acinetobacter, and Pseudomonas bacterial strains. Initial growth rate curves for the bacterial strains were determined so that consistent infection with 1×10⁹ colony forming units (cfus) can be achieved.

Skin samples are incubated for a 3 hour period to allow infection growth followed by NORS treatment. Treatment volume, dose and exposure time are determined prior to each experimental set. Following NORS treatment tissues are homogenized and serial dilutions are plated on Lysogeny Broth (LB) agar and colonies counted following 24 hours of incubation at 37° C. Additionally, pH and nitrite/nitrate levels are measured and recorded for statistical analyses. Parallel controls are implemented and all experiments are done in triplicate (a,b,c).

A NORS solution is prepared by addition of sodium nitrite and citric acid to a saline solution. Nitrite concentration of the NORS is 100 mM and pH is adjusted to 3.6. Approximately 1 mL of NORS is administered to the skin samples and the samples are incubated for 30 minutes followed by further processing as previously described along with a control. The following bacterial counts result:

| dilution | Control | a | b | c |
|---|---|---|---|---|
| 10⁻⁷ | 9 | 0 | 0 | 0 |
| 10⁻⁶ | 60 | 0 | 0 | 0 |
| 10⁻⁵ | TNTC | 0 | 1 | 2 |
| 10⁻⁴ | TNTC | 19 | 23 | 26 |
| 10⁻³ | TNTC | 220 | TNTC | TNTC |
| 10⁻² | TNTC | TNTC | TNTC | TNTC |
| 10⁻¹ | TNTC | TNTC | TNTC | TNTC |

### Example 3

### Safety of NORS to Treat Mastitis

### Procedure

Three cows were tested, labeled 1064, 1108, and 1111, respectively. Exposure to experimental interventions totaled 12 days. NORS was administered as a single treatment once every 48 hours, with an increase in NORS dose each time. Cows were milked twice daily as per regular farm management. Following regular milk out with the automated machines, the following steps were performed:

Day 1—a. Intramammary infusion of 40 ml of saline after residual milk was manually stripped out of the teat.
b. Immediate stripping of all saline/milk from teat.
c. Intramammary infusion of 40 ml of saline at pH 3.7.
d. After 5 min, stripping all saline/milk from teat.
   Day 2—Intramammary infusion of 50 mM NORS (40 ml ) at pH 3.7 after residual milk was manually stripped out of the teat.
   Day 4—Intramammary infusion of 100 mM NORS (40 ml ) at pH 3.9 after residual milk was manually stripped out of the teat.
   Day 6—Intramammary infusion of 200 mM NORS (40 ml ) at pH 3.9 after residual milk was manually stripped out of the teat.
   Day 8—Intramammary infusion of 400 mM NORS (40 ml ) at pH 3.9 after residual milk was manually stripped out of the teat.

### Testing

### Day 1 Evening

stripping
Intramammary infusion of 40 ml of saline after residual milk was manually stripped out of the teat.
Immediate stripping of all saline/milk from teat.
Intramammary infusion of 40 ml of saline at pH 3.7.
After 5 min, stripping all saline/milk from teat.

### Day 2 Morning:

stripping
Intramammary infusion of 50 mM NORS (40 ml ), after residual milk was manually stripped out of the teat.
5 min, 30 min, 8 hrs—blood

### Day 2 Evening:

Milking: 50 ml were stripped manually from treated quarter plus 10 ml from each of the other 3 sections, followed by machine milking (sample B), and manually stripping again 20 ml from treated quarter (sample C). Samples were mixed with ethanol and tested for nitrites.
Blood sample (for nitrites measurements and MetHg). Taken with heparin tube and transferred to two 1.5 ml tubes. One tube for immediate MetHg measurement and the other was spin down for serum. Serum was divided to 2 tubes and into freezer for nitrites and nitrates measurements.
   Day 3 Milk sample were tested in the morning—50 ml sample from treated quarter.

### Day 4 Morning:

stripping
Intramammary infusion of 100 mM NORS (40 ml ), after residual milk was manually stripped out of the teat.
5 min, 30 min, 8 hrs—blood

### Day 4 Evening:

Milking: 50 ml were stripped manually from treated quarter plus 10 ml from each of the other 3 sections, followed by machine milking (sample B), and manually stripping again 20 ml from treated quarter (sample C). Samples were mixed with ethanol and tested for nitrites.
Blood sample (for nitrites measurements and MetHg). Taken with heparin tube and transferred to two 1.5 ml tubes. One tube for immediate MetHg measurement and the other was spin down for serum. Serum was divided to 2 tubes and into freezer for nitrites and nitrates measurements.
   Day 5 Milk sample were tested in the morning—50 ml sample from treated quarter

### Day 8 Morning:

stripping
Intramammary infusion of 200mM NORS (40 ml ), after residual milk was manually stripped out of the teat.
5 min, 30 min, 8 hrs—blood

### Day 8 Evening:

Milking: 50 ml were stripped manually from treated quarter plus 10 ml from each of the other 3 sections, followed by machine milking (sample B), and manually stripping again 20 ml from treated quarter (sample C). Samples were mixed with ethanol and tested for nitrites.
Blood sample (for nitrites measurements and MetHg). Taken with heparin tube and transferred to two 1.5 ml tubes. One tube for immediate MetHg measurement and the other was spin down for serum. Serum was divided to 2 tubes and into freezer for nitrites and nitrates measurements.
   Day 9 Milk sample were tested in the morning—50 ml sample from treated quarter.

### Day 10 Morning:

stripping
Intramammary infusion of 400 mM NORS (40 ml ), after residual milk was manually stripped out of the teat.
5 min, 30 min, 8 hrs—blood

### Day 10 Evening:

Milking: 50 ml were stripped manually from treated quarter plus 10 ml from each of the other 3 sections, followed by machine milking (sample B), and manually stripping again 20 ml from treated quarter (sample C). Samples were mixed with ethanol and tested for nitrites.
Blood sample (for nitrites measurements and MetHg). Taken with heparin tube and transferred to two 1.5 ml tubes. One tube for immediate MetHg measurement and the other was spin down for serum. Serum was divided to 2 tubes and into freezer for nitrites and nitrates measurements.
   Day 11 Milk sample were tested in the morning—50 ml sample from treated quarter.

### Results

As a preliminary observation, there was no obvious effect on behavior, general well-being, brightness, hydration, or boumatic activity. Additionally, no curdling of the milk was seen.

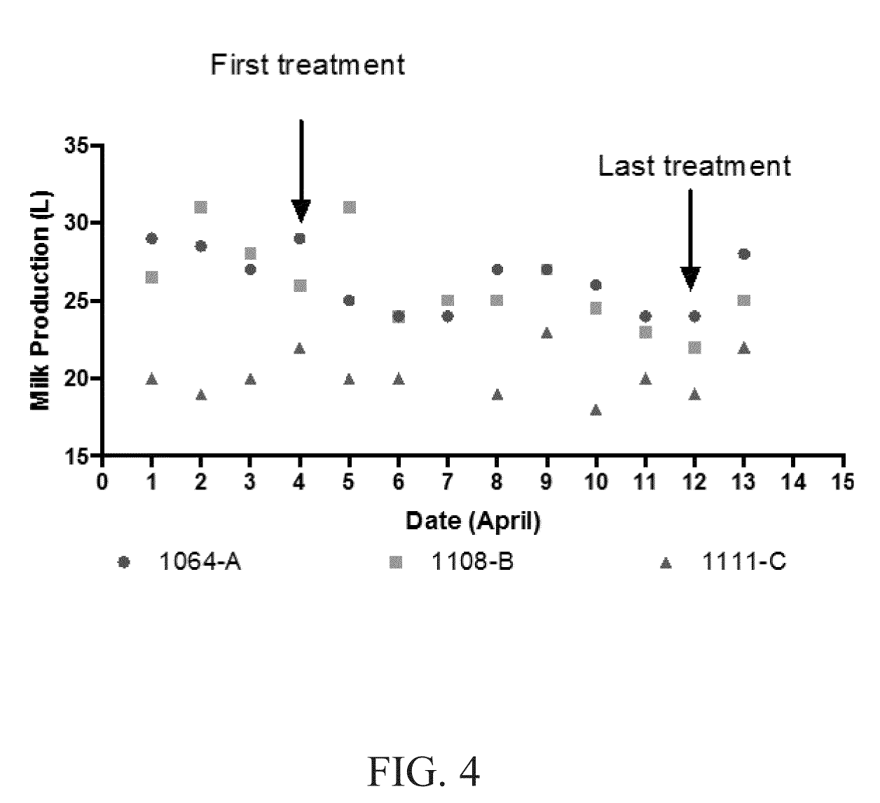

FIG. 4 shows milk production levels for each cow on each day of the treatment period. There seems to be a trend in reduction of milk production in animal 1108 but not in the other two animals. It is unclear whether this is related to the treatment.

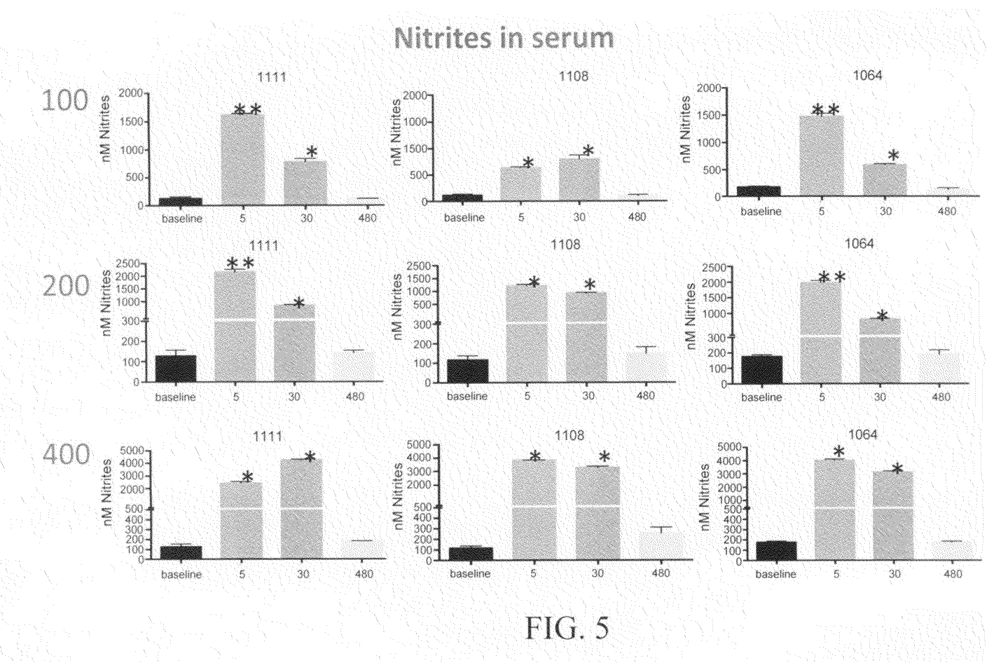

As can be seen in FIG. 5, there was a rise in serum nitrites in the samples taken 5 and 30 min post treatment. All values returned to baseline at samples taken 8 hours post treatment.

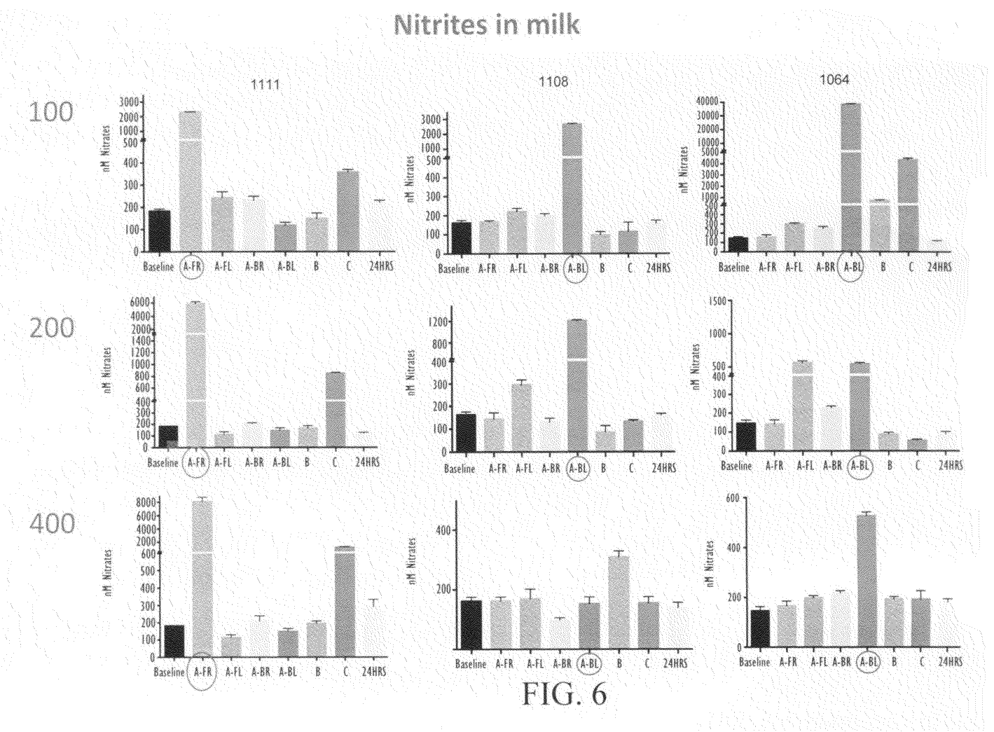

It is very clear from the measurements of nitrites in milk that there was a significant rise in the amount of nitrites in the milk 10 hours post treatment. The rise was seen predominantly in the quarter that was treated. The concentration of nitrites in the milk, including in the treated section, returned to baseline at or before 24 hours post treatment. This data is shown in FIG. 6.

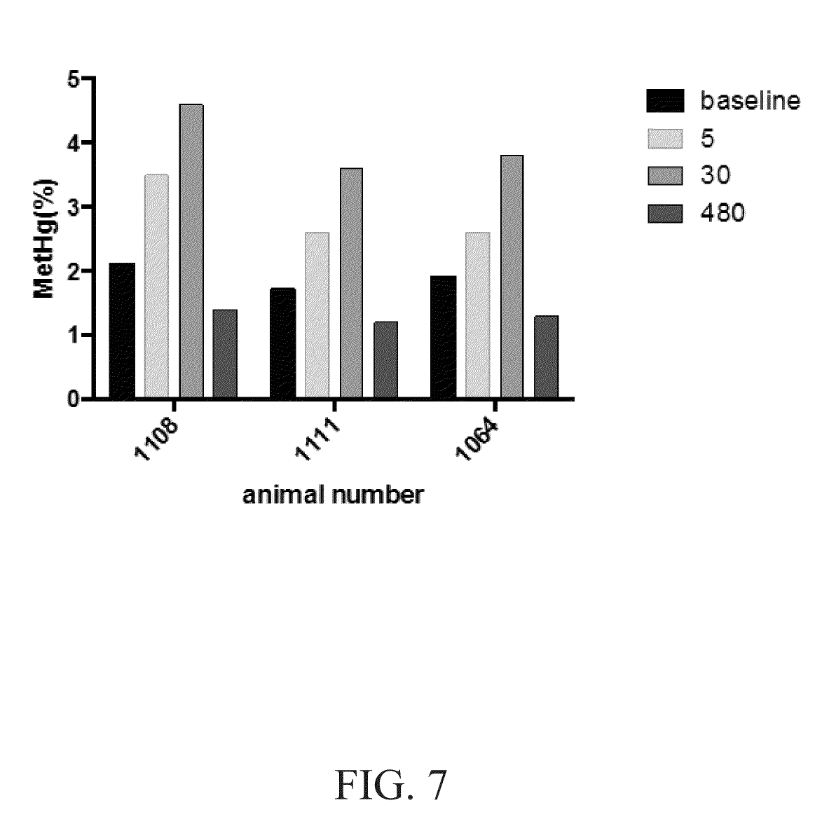

As can be seen in FIG. 7, there was a rise in MetHg(%) in the blood samples taken 5 and 30 min post treatment with the highest concentration (no rise was detected after treating with lower concentrations). This rise is still within the safety levels (<10%). This shows that the nitrites did get a systemic effect but 8 hours post treatment (and probably earlier) all values returned to baseline.

The treatment of NORS at a strength of up to 400 mM proved to be safe to the animals with no adverse events or curdling of milk. The treatment at highest concentration showed some systemic evidence, measured by a rise of 2-2.5% of MetHg(%) in blood 30 minutes post treatment. This effect was short acting and all values returned to baseline during the 8 hours post treatment measurements. Nitrites were also found in serum, but also returned to baseline by 8 hours. An increase in both nitrites and nitrates was detected in the milk, mostly in the treated quarter, at 10 hours post milking. All nitrites levels in the milk returned to baseline in the 24 hours post treatment milk samples. This may suggest that a withhold period after treatment need only be 24 hours or less. Nitrates levels were all below the normal milk nitrates levels presented in the literature within 24 hours post milking. From this study it seems that treating animals with up to 400 mM at pH 3.9 of NORS is safe.

### Example 4

### In vitro Milk Studies

### Procedure

Prepared nitrite solutions at 100-400 mM pH 3.2-4.0. Mixed the following ratios of milk to nitrite solution:
2 to 1 (660 μl milk+330 μl nitrite)
1 to 1 (500 μl milk+500 μl nitrite)
1 to 2 (330 μl milk+660 μl nitrite)
1 to 3 (250 μl milk+740 μl nitrite)
1 to 3 saline control (200 μl milk+790 μl saline)

After mixing the milk and nitrite solutions, the various samples were observed to determine whether any curdling of the milk occurred. Subsequently, 10⁵ CFU/ml E.coli was added to each sample. Samples were incubated 1-10 min and then plated.

### Results

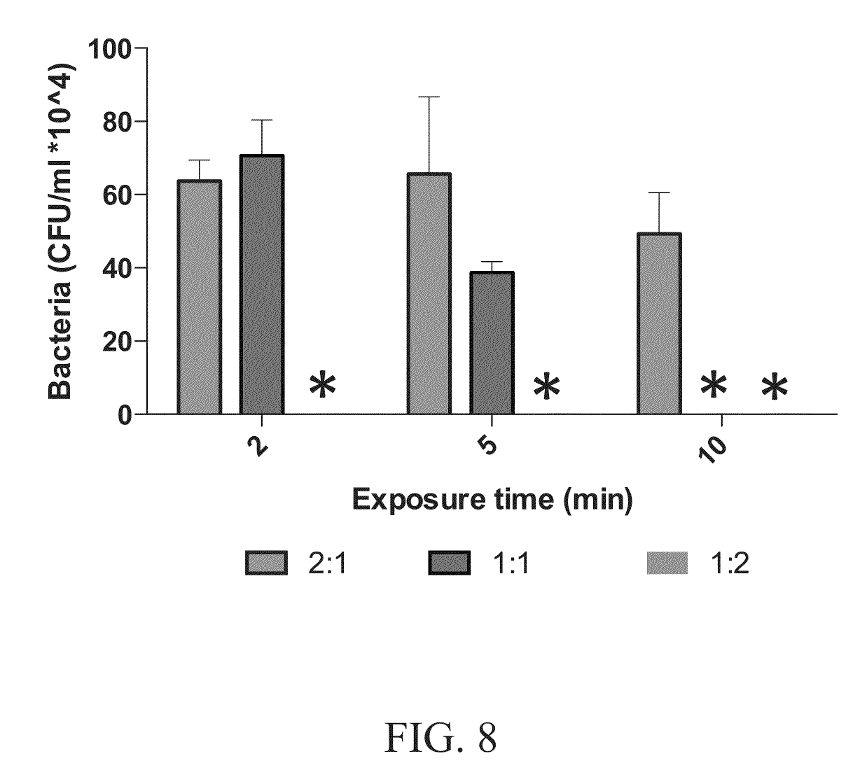

The 100 mM samples at pH 3.2 caused curdling at 1:1 and 2:1 milk to nitrites. The 2× mM samples at pH 3.2 caused curdling at all ratios. However, the 200 mM sample at pH 3.8 caused no curdling at the 1:1 ratio and caused 70% reduction in E. coli counts after 5 min and complete kill within 10 min. Additionally, the 400 mM sample at pH 4 also caused no curdling and there was a complete eradication of E. coli at a ratio of 1:2 at 2 min. E. coli counts in the 400 mM samples at ratios of 1:2, 1:1, and 2:1 milk to nitrite solution are shown in FIG. 8.

While these invention embodiments and examples have been with reference particularity, it is apparent that other embodiments and variations of those provided may be devised by others skilled in the art without departing from the spirit and scope of this disclosure. The appended claims are intended to be construed to include all such embodiments and equivalent variations.

What is claimed is:

1. A method of treating a mammary condition in a subject, comprising administering a therapeutically effective amount of gaseous nitric oxide (gNO) to the subject's mammary tissue from a nitric oxide releasing solution (NORS).

2. The method of claim 1, wherein the mammary tissue is an internal tissue, including mammary cisterns, milk ducts, lobules and alveoli.

3. The method of claim 1, wherein the NORS is prepared from a two-part composition, a first part of the two-part composition including at least one nitrite or salt thereof and a second part of the two-part composition including an acidifying agent.

4. The method of claim 1, wherein the NORS includes at least one nitric oxide releasing compound in an amount of less than about 1.0% w/v.

5. The method of claim 1, wherein the NORS is administered in an amount from about 0.25 mL to about 50 mL.

6. The method of claim 1, wherein the NORS is prepared within about 10 minutes of administration to the mammal.

7. The method of claim 1, wherein the NORS is administered post-milking or post-nursing.

8. The method of claim 1, wherein mammary condition is at least one of mastitis, mammary herpes, mammary tuberculosis, mammary abscess, mammary syphilis, poxvirus infection, dermatitis, warts, mammary actinomycosis, and duct ectasia.

9. The method of claim 1, wherein mammary condition is mastitis.

10. The method of claim 1, wherein administration occurs when disease state is present.

11. The method of claim 1, wherein administration is prophylactic.

12. The method of claim 1, wherein administration is intramammary delivery.

13. The method of claim 1, wherein the therapeutically effective amount of gNO is a concentration of from about 50 ppm to about 400 ppm.

14. The method of claim 1, wherein serum nitrite levels induced by administration of the NORS return to baseline within a period of up to 480 minutes post administration.

15. The method of claim 1, wherein blood MetHg(%) levels induced by administration of NORS return to baseline within a period of up to 480 minutes post administration.

16. A system for treating a mammary condition in a subject as recited in claim 1, comprising:
a first component configured to release gNO upon acidification thereof;
a second component configured to acidify the first component; and
a device for administering the first and second component such that when combined, the components form a composition that releases a therapeutically effective amount of gNO to the subject.

17. The system of claim 16, wherein the system further comprises a device for combining the first and second components either prior to, during, or following administration to the subject.

18. The system of claim 28, wherein the device is configured to administer the first and second component within a mammary cistern.

19. The system of claim 16, wherein the first and second components are combined prior to administration.

20. The system of claim 16, wherein the first and second components are combined upon administration.

21. The system of claim 16, wherein the first and second components are combined following administration.

22. The system of claim 16, wherein the first component includes at least one nitrite or salt thereof and the second component includes an acidifying agent.

23. The system of claim 16, wherein the first and second component are combined to form an activated composition.

24. The system of claim 36, wherein the volume of activated composition is from about 10 to about 50 ml.

25. The system of claim 23, wherein the pH of activation is lower than about 5.0.

26. The system of claim 23, wherein the pH of activation is from about 3.2 to about 4.2.

27. The system of claim 23, wherein the activated formulation releases gNO at a concentration of from about 50 to about 400 ppm.

28. A method for minimizing an interruption in usable milk production by a cow due to treatment of the cow for a mammary condition, comprising:
administering to the cow's mammary tissue, a therapeutically effective amount of gNO from a NORS solution.

29. The method of claim 28, wherein the interruption occurs for less than 6 milking events.

30. The method of claim 28, wherein the interruption occurs for no more than 1 milking event.

31. The method of claim 28, wherein the mammary condition is mastitis.

32. The method of claim 28, wherein the interruption is for a period of up to 48 hours.
